(19) United States
(12) Patent Application Publication
(10) Pub. No.: US 2013/0337062 A1
(43) Pub. Date: Dec. 19, 2013

(54) GASTRO-RESISTANT ENZYME PHARMACEUTICAL COMPOSITIONS

(71) Applicant: Aptalis Pharma Canada Inc., (US)

(72) Inventors: Mircea Alexandru MATEESCU, Mont-Saint-Hilaire (CA); Ingry Janet BUSTOS, Mont-Saint-Hilaire (CA); Yves DUMOULIN, Ste-Julie (CA); Pompilia Ispas SZABO, Quebec (CA)

(73) Assignee: Aptalis Pharma Canada Inc., Mont-Saint-Hilaire (CA)

(21) Appl. No.: 13/874,903

(22) Filed: May 1, 2013

Related U.S. Application Data
(63) Continuation of application No. 13/618,383, filed on Sep. 14, 2012, now abandoned.
Application No. 13/618,383 is a continuation of application No. PCT/IB2011/000579, filed on Mar. 18, 2011.
(60) Provisional application No. 61/315,814, filed on Mar. 19, 2010.

(51) Int. Cl. A61K 38/46; A61K 9/00

(52) U.S. Cl. CPC A61K 38/46; A61K 9/0053
USPC 424/474; 435/188; 424/94.2; 424/464

(57) ABSTRACT

The present invention generally relates to compacted pharmaceutical compositions (such as tablets) comprising one or more enzymes, where the composition is monolithic or multiparticulates (such as mini-tablets, micro-tablets, or prills), or where the composition has multiple layers with the outermost layer containing one or more enzymes.

This application claims the benefit of U.S. Provisional Application No. 61/315,814, filed Mar. 19, 2010, which is hereby incorporated by reference.

### FIELD OF THE INVENTION

The present invention generally relates to pharmaceutical compositions (such as tablets) comprising one or more enzymes (for instance, pancreatic enzymes), where the composition is monolithic or a single layer of multiparticulates (such as mini-tablets, micro-tablets, or prills), or where the composition has multiple layers with the outermost layer containing one or more enzymes.

### BACKGROUND

Various disease states of the pancreas produce a condition in which insufficient pancreatic enzymes are available for digestive processes. Enzyme deficiency associated with, for example, pancreatitis, pancreatectomy, steatorrhea, and cystic fibrosis, can disrupt the breakdown and absorption of nutrients resulting in malnutrition.

Exogenously administered pancreatic enzymes can be used to treat pancreatic insufficiency. Pancreatic enzymes exhibit optimal activity at near neutral pH conditions found in the small intestine. Under gastric conditions, these orally administrated enzymes generally become irreversibly inactivated.

Several delayed release forms of orally administered pancreatic enzymes have been proposed. Pancreatic enzymes can be formulated as gastric resistant microspheres (See U.S. Pat. Nos. 6,051,220; 5,405,621; 5352,460; 5,324,514, and 5,260,074). Such compositions may be resistant to gastric fluids, but fail to exhibit satisfactory release profiles. For example, enteric coated preparations often dissolve too late in the upper intestine to make the enzymes unavailable at the desired location. Further, enteric-coated compositions are often unable to release active enzyme in patients with exocrine pancreas insufficiency because the upper regions of the small intestine in these patients is often acidic. See Barraclough M, Taylor C J., Twenty-four hour ambulatory gastric and duodenal pH profiles in cystic fibrosis: effect of duodenal hyperacidity on pancreatic enzyme function and fat absorption, J Pediatr Gastroenterol Nutr 1996, 23: 45-50; Carriere F, Grandval P, Renou C, et al., Quantitative study of digestive enzyme secretion and gastrointestinal lipolysis in chronic pancreatitis, Clin Gastroenterol Hepatol 2005, 3: 28-38; Youngberg C A, Berardi R R, Howatt W F et al., Comparison of gastrointestinal pH in cystic fibrosis and healthy subjects, Dig Dis Sci 1987, 32: 472-80; Zentler-Munro P L, Fitzpatrick W J, Batten J C, Northfield T C, Effect of intrajejunal acidity on aqueous phase bile acid and lipid concentrations in pancreatic steatorrhoea due to cystic fibrosis, Gut 1984, 25: 500-7.

Compositions comprising cross-linked enzyme preparations are known (See U.S. Patent Publication Nos. 2001/0046493 and 2003/0017144). Cross-linking has been shown to enhance resistance to acidic pH. However, the efficient preparation of cross-linked proteins is difficult, and the cross-linking process may adversely affect enzyme activity. Furthermore, crosslinking enzymes may result in difficulties in obtaining regulatory approval, and difficulties in the production of compliant proteins. Compositions comprising fungal and microbial enzyme mixtures as an alternative to animal enzymes for treating pancreatic insufficiency have also been disclosed (See U.S. Pat. No. 6,051,220, and U.S. Patent Publication Nos. 2008/0279839 and 2004/0057944).

Currently, orally administrable pancrelipase dosage forms are prescribed for pancreatic insufficiency. Patients, however, must swallow several of these dosage forms each day. In many cases, patients may be required to swallow 8 or more dosage forms daily. Patient compliance can be increased by reducing the high number of dosage forms which must be administered.

Accordingly, there remains a need for improved enzyme preparations for treating disorders related to pancreatic enzyme deficiency.

### SUMMARY OF THE INVENTION

The present inventors surprisingly discovered that compacted, uncoated tablets of enzymes (such as pancrelipase) retain significant enzymatic activity even after exposure to simulated gastric fluids. In the case of pancrelipase preparations, reduction or exclusion of typical excipients, such as enteric coatings, can result in approximately 20-40% reduction in size. Alternatively, the drug load of the preparations can be significantly increased without a similar increase in size, thus reducing the number of dosage forms a patient must swallow each day for the same dose of enzymes

In the compacted compositions of the present invention, the enzymes (such as pancreatic enzymes) act as active ingredients as well as a binder and a pH-sensitive gel-forming agent. One embodiment of the present invention is a compacted pharmaceutical composition comprising one or more enzymes (e.g., pancrelipase) self-assembled such that the enzymes have greater cohesive inter-particular strength after compaction than prior to compaction. The composition is typically orally administrable, and can be a tablet or multiparticulates (such as mini-tablets, micro-tablets, or prills), for which one or multiple units can be eventually incorporated into, for example, a capsule. The composition is typically gastroresistant. In one preferred embodiment, the tablet shape in simulated gastric fluid (SOF) is substantially maintained. Without being bound by any particular theory, the inventors believe that upon administration, an outer layer is formed (as shown for instance in FIG. 1) which contributes to gastro-resistance of the dosage form. The inventors have also found that the inner part of the tablet is substantially dry (FIG. 1). Preferably, the pharmaceutical compositions retain at least about 30, about 40, about 50, about 60, about 70, about 80, or about 90% of their activities in the inner dry core of the pharmaceutical composition after exposure to simulated gastric fluid for 1 or 2 hours. Because of the enhanced gastro-resistance of the compositions of the present invention, the drug content of the composition can be about 80, about 90, about 95, or even about 99% or greater (based on the total weight of the composition).

The enzymes can be digestive hydrolases. In one embodiment, the enzymes are selected from amylases, lipases, proteases, and any combination of any of the foregoing. In one preferred embodiment, the composition contains pancrelipase. The enzymes can be of porcine or non-porcine origin. For instance, the pancrelipase can be of porcine origin.

In a preferred embodiment, the pharmaceutical composition is un-coated. In another preferred embodiment, the pharmaceutical composition is monolithic. Yet, another preferred embodiment consists is an un-coated monolithic dosage form, such as an un-coated monolithic tablet. The pharmaceutical composition can be formed by compaction at a force of from about 0.25 to about 3.0 T.

Preferably, the composition is substantially free (e.g., contains less than about 5, about 4, about 3, about 2, about 1, about 0.5, or about 0.2% w/w) of binder and/or disintegrant, or completely free of binder and/or disintegrant. In one embodiment, the composition is substantially free of binder and substantially free of disintegrant. In another embodiment, the composition is substantially free of binder and free of disintegrant. In yet another embodiment, the composition is free of binder and substantially free of disintegrant.

Preferably, the composition is substantially free (e.g., contains less than about 5, about 4, about 3, about 2, about 1, about 0.5, or about 0.2% w/w) of excipients, or completely free of excipients.

According to one preferred embodiment, the composition (e.g., tablet) is not enterically coated.

Another embodiment is a monolithic, compacted, gastro-resistant pharmaceutical composition comprising one or more enzymes self-assembled so as to enhance cohesion within the composition. The composition is typically orally administrable, and can be a tablet, or a mini-tablet or multiparticulates such as prills which can be incorporated into, for example, a capsule. The enzymes can be any described in the present application, such as pancrelipase. Furthermore, the composition can have a drug content of at least about 65, about 80, about 90, about 95, or about 99% or greater, or can have a drug content of 100% by weight. In addition, other pharmaceutically active ingredients can be incorporated to obtain multipurpose pharmaceutical dosage forms. Preferably, the composition is substantially free of excipients, or completely free of excipients. According to one preferred embodiment, the composition is not enterically coated.

Another embodiment is a monolithic, compacted, gastro-resistant pharmaceutical composition comprising pancrelipase. The pancrelipase comprises a mixture of lipase, amylase, and proteases. The composition is typically orally administrable, and can be a tablet or multiparticulates (such as mini-tablets, micro-tablets, or prills), for which one or multiple units can be eventually incorporated into, for example, a capsule. After administration, an outer coating is formed from the enzymes exposed on the surface of the composition. The lipases, amylases, and proteases in the inner core composition preferably retain at least about 30% of their activity, after the composition is exposed to simulated gastric fluid for 1 hour or 2 hours.

In the inner (dry) core of the compositions described above, the lipases and amylases preferably retain at least about 80% and about 30% of their activity, respectively, after exposure to simulated gastric fluid for 2 hours. The proteases in the composition preferably retain at least about 70% of its activity after exposure to simulated gastric fluid for 1 hour.

More preferably, the lipase retains at least about 40, about 50, about 60, about 70, about 80, or about 90% of its activity in the inner core of the composition, after exposure to simulated gastric fluid for 1 hour or 2 hours. The amylase more preferably retains at least about 40, about 50, or about 60% of its activity, after exposure to simulated gastric fluid for 1 hour or 2 hours. The proteases more preferably retain at least about 40, about 50, about 60, about 70, or about 80% of its activity, after exposure to simulated gastric fluid for 1 hour. These data can be obtained by exposing the compositions (e.g., tablets) to a particular volume of SGF or SIF (see, for instance, the testing method below).

The composition can be directly compacted with a compression force of from about 0.25 to about 3.0 T.

The composition can have a drug load of about 80, about 90, about 95, or even about 99% by weight or greater.

Preferably, the composition is substantially free of excipients, or completely free of excipients.

According to one preferred embodiment, the composition is not enterically coated.

Yet another embodiment is a compacted pharmaceutical composition comprising one or more enzymes, where the composition has an enzyme drug load of at least about 80%. Preferably, the composition has a drug content of at least about 90, about 95, or about 99% or greater. The composition is typically orally administrable, and can be a tablet or multiparticulates (such as mini-tablets, micro-tablets, or prills), for which one or multiple units can be eventually incorporated into, for example, a capsule. The enzymes can be any described in the present application, such as pancrelipase. According to one preferred embodiment, the composition is not enterically coated.

Yet another embodiment is a monolithic, compacted, gastro-resistant pharmaceutical composition comprising one or more enzymes self-assembled so as to enhance cohesion within the composition. The composition is typically orally administrable, and can be a tablet or multiparticulates (such as mini-tablets, micro-tablets, or prills), for which one or multiple units can be eventually incorporated into, for example, a capsule. The enzymes can be any described in the present application, such as pancrelipase. Preferably, the composition has a drug content of at least about 80, about 90, about 95, or about 99% or greater. Preferably, the composition is substantially free of excipients, or completely free of excipients. According to one preferred embodiment, the composition is not enterically coated.

Yet another embodiment is a compacted pharmaceutical composition comprising one or more enzymes, wherein the composition is substantially free (or completely free) of excipients and is not enterically coated. The composition is typically orally administrable, and can be a tablet or multiparticulates (such as mini-tablets, micro-tablets, or prills), for which one or multiple units can be eventually incorporated into, for example, a capsule. The enzymes can be described in the present application, such as pancrelipase. Preferably, the composition has a drug content of at least about 80, about 90, about 95, or about 99% by weight or greater.

Yet another embodiment is a multi-layer, compacted pharmaceutical composition comprising one or more enzymes in the outermost layer of the composition. The composition is typically orally administrable, and can be a tablet or multiparticulates (such as mini-tablets, micro-tablets, or prills), for which one or multiple units can be eventually incorporated into, for example, a capsule. Preferably, the enzymes are self-assembled such that the enzymes have greater cohesive strength resulting from the compaction. The composition is preferably gastroresistant. In one embodiment, one or more of the enzymes retain at least about 30, about 40, about 50, about 60, about 70, about 80, or about 90% of their activity in the inner tablet core after exposure to simulated gastric fluid for 1 hour.

Yet another embodiment is a pharmaceutical composition comprising a layer of one or more enzymes, wherein the layer is substantially free of binder and/or disintegrant.

Yet another embodiment is a pharmaceutical composition consisting of pancrelipase, wherein the lipase of the pancrelipase retains at least about 80% of its activity after exposure to pH of 1.2 at 37° C. for 2 hours. In a preferred embodiment, the lipase of the pancrelipase retains at least about 85 or about 90% of its activity (e.g., in the inner dry core of the pharmaceutical composition) after exposure to pH of 1.2 at 37° C. for 2 hours. In one embodiment, the amylase and/or protease in the pharmaceutical composition retain at least about 30, about 40, about 50, about 60, about 70, about 80, or about 90% of their activities in the inner dry core of the pharmaceutical composition after exposure to pH of 1.2 at 37° C. for 2 hours.

Yet another embodiment is a pharmaceutical composition consisting of pancrelipase obtainable by compressing pancrelipase free of other excipients at a compression force of from about 0.25 to about 3.0 T (e.g., from about 1.0 to about 3.0 T or from about 1.25 to about 3.0 T).

In any of the aforementioned embodiments, the composition may comprise from about 1,000 to about 150,000 USP units of lipase, from about 3,000 to about 300,000 U proteases, and from about 3,000 to about 500,000 U amylases. In another embodiment, the composition comprises from about 2,000 to about 75,000 USP units of lipase, from about 8,000 to about 250,000 U proteases, and from about 8,000 to about 250,000 U amylases. In yet another embodiment, the composition comprises from about 2,000 to about 40,000 USP units of lipase, from about 8,000 to about 160,000 U proteases, and from about 8,000 to about 160,000 U amylases.

Yet another embodiment is a process for preparing a pharmaceutical composition comprising one or more enzymes. The method includes compacting an enzyme preparation free or substantially free of excipients. Preferably, the compaction is performed at a compression force of from about 0.25 to about 3.0 T. According to one preferred embodiment, the compacted pharmaceutical composition is a tablet. According to one particular embodiment, the pharmaceutical composition is not enterically coated.

Yet another embodiment is a method for treating a digestive disorder by administering a pharmaceutical composition of the present invention. Preferably, a therapeutically effective amount of the pharmaceutical composition is administered. Preferably, the composition is orally administered.

In one embodiment, the composition comprises pancrelipase. The patient may suffer from partial or complete exocrine pancreas insufficiency. The exocrine pancreas insufficiency may be concomitant with cystic fibrosis, chronic pancreatitis, post-pancreatectomy, post-gastrointestinal bypass surgery (e.g., Billroth II gastroenterostomy), ductal obstruction from neoplasm (e.g., of the pancreas or common bile duct), alcoholism, or pancreatic carcinomas.

Yet another embodiment is a method for controlling steatorrhea by administering to a patient in need thereof a pharmaceutical composition of the present invention, where composition comprises pancrelipase. Preferably, the composition is orally administered.

The inventors of the present invention have discovered that enzyme preparations become gastro-resistant upon compaction. Without being bound by any particular theory, the inventors describe in this and the next paragraph the theorized mechanism by which the present invention is believed to operate. The inventors believe that the enzymes undergo self-assembly during the compaction process. The self-assembly results from various types of interactions between protein chains, such as hydrogen associations (e.g., from histidine, lysine, tyrosine, and serine), other associative binding (e.g., π-π interactions involving aromatic rings of phenylalanine and tyrosine), and ionic interactions (e.g., —COO⁻ with ⁺NH₃ between glutamate-lysine and aspartate-lysine). These interactions also improve the stability of the shape of the pharmaceutical composition (e.g., tablet). Furthermore, the ionic stabilization results in the protein acting as a buffer and thus enhances gastric stability.

The associative and ionic interactions are pH sensitive. The pharmaceutical compositions exhibit strong cohesion in acidic pH (and thus afford gastric stability). In intestinal fluid, however, the carboxylic groups are deprotonated, which triggers hydration, erosion of the pharmaceutical composition, and disintegration with the release of the therapeutic enzymes. The enzymes thus act as a biologically active agent as well as a binder and pH sensitive swelling agent.

Because the compacted pancrelipase itself acts as a binder and is gastro-resistant, a tablet with a significantly higher drug content can be obtained. Thus, either a significantly larger amount of therapeutic enzyme can now be delivered in tablets of the same size as prior art tablets, or smaller tablets having the same amount of drug as prior art tablets can be used. Furthermore, in many embodiments, an enteric coating is not necessary to protect the enzyme from gastric acidity.

### BRIEF DESCRIPTION OF THE DRAWINGS

FIG. 1 is an image of the cross-section of a non-enterically coated pancreatic enzyme concentrate (PEC) tablet with a self-coating formed after exposure to simulated gastric fluid for 1 hour.

FIG. 2 shows the thickness of the hydrated layer in tablets, prepared by the procedure described in Example 1 having the sizes indicated in Table XII, after exposure to SGF.

### DETAILED DESCRIPTION

As used herein, the term “comprising” is open ended and, in connection with a composition, refers to the elements recited. The term “comprising” as used in connection with the compositions described herein can alternatively cover compositions “consisting essentially of” or “consisting of” the recited components (e.g., pancrelipase).

As used herein, the term “enzymes” refers to any polypeptide having catalytic activity. Generally, enzymes may be available in powder or crystalline form, typically as enzyme concentrates derived from animal sources. However, plant and microbial derived systems can also be used. Non-limiting examples of enzymes include digestive enzymes.

Digestive enzymes include, for example, lipases, amylases and proteases. In one embodiment, the digestive enzyme is pancrelipase. Pancrelipase (or “pancreatin”) typically includes amylase, lipase, and protease enzymes. Non-limiting examples of digestive enzymes also include lipase and co-lipase, trypsin, chymotrypsin, chymotrypsin B, pancreatopeptidase, carboxypeptidase A, carboxypeptidase B, glycerol ester hydrolase, phospholipase, sterol ester hydrolase, elastase, kininogenase, ribonuclease, deoxyribonuclease, α-amylase, papain, chymopapain, glutenase, bromelain, ficin, β-amylase, cellulase, P-galactosidase, lactase, sucrase, isomaltase, and any combination of any of the foregoing. Other non-limiting examples of digestive enzymes include exogenous enzymes such as β-amylase, cellulase, and any combination of any of the foregoing.

In one embodiment, the digestive enzyme is a pancreatic enzyme. The term “pancreatic enzyme” refers to any one of the enzyme types present in the pancreatic secretion, such as amylase, lipase, protease, or mixtures thereof, or any extract of pancreatic origin having enzymatic activities, such as pancreatin. The pancreatic enzyme can be obtained through extraction from the pancreas (e.g., of porcine or non-porcine origin), produced artificially, or obtained from sources other than the pancreas, such as from microbes, plants or other animal tissues.

In another embodiment, the digestive enzyme comprises a lipase. The term “lipase” refers to an enzyme that catalyzes the hydrolysis of lipids to glycerol and simple fatty acids. Examples of lipases include, but are not limited to, animal lipase (e.g., porcine lipase), bacterial lipase (e.g., Pseudomonas lipase and/or Burkholderia lipase), fungal lipase, plant lipase, recombinant lipase, chemically-modified lipase, or mixtures thereof.

In yet another embodiment of the present invention, the digestive enzyme comprises an amylase. The term “amylase” refers to glycoside hydrolase enzymes that break down starch, for example α-amylases, β-amylases, γ-amylases, acid α-glucosidases, salivary amylases such as ptyalin. The amylases suitable for use in the compositions of the present invention include, but are not limited to, animal amylases, bacterial amylases, fungal amylases, plant amylases, recombinant amylases, and chemically modified amylases, or mixtures thereof.

In another embodiment, the digestive enzyme comprises proteases. The term “proteases” refers to enzymes that degrade peptide bonds. Proteases are generally identified by their catalytic type, e.g., aspartic acid peptidases, cysteine (thiol) peptidases, metallopeptidases, serine peptidases, threonine peptidases, alkaline or semi-alkaline proteases, neutral and peptidases of unknown catalytic mechanism. Non-limiting examples of proteases include serine proteases, threonine proteases, cysteine proteases, aspartic acid proteases (e.g., plasmepsin) metalloproteases, and glutamic acid proteases. Proteases suitable for use in the compositions of the present invention include, but are not limited to animal proteases, bacterial proteases, fungal proteases (e.g., an Aspergillus melleus protease), plant proteases, recombinant proteases, and chemically modified proteases, or mixtures thereof.

In one embodiment, the digestive enzyme is a porcine pancreatic extract comprising various lipases (e.g., lipase and phospholipase A2), proteases (e.g., trypsin, chymotrypsin, carboxypeptidase A and B, elastase, and kininogenase), amylases, and optionally nucleases (ribonuclease, deoxyribonuclease), cholesterol esterase, and cofactors such as colipase. In another embodiment, the digestive enzyme is substantially similar to human pancreatic fluid. In yet another embodiment, the digestive enzyme is non-porcine pancrelipase. In yet another embodiment, the digestive enzyme is pancrelipase of porcine origin. In another embodiment, the digestive enzyme is pancrelipase USP. In still another embodiment, the digestive enzyme is pancrelipase having a lipase activity of from about 69 to about 120 U USP/mg, amylase activity of greater than or equal to about 216 U USP/mg, protease activity of greater than or equal to about 264 U USP/mg, and total protease activity of greater than or equal to about 264 U USP/mg.

In one embodiment, the compositions of the present invention can comprise one or more lipases (i.e., one lipase, or two or more lipases), one or more amylases (i.e., one amylase, or two or more amylases), one or more proteases (i.e., one protease, or two or more proteases), mixtures of one or more lipases and colipase with one or more amylases, mixtures of one or more lipases with one or more proteases, mixtures of one or more amylases with one or more proteases, or mixtures of one or more lipases with one or more amylases and one or more proteases.

Lipase activities in the compositions of the present invention can range from about 1,000 to about 150,000 International Units (U). Amylase activities in the compositions of the present invention can range from about 3,000 to about 500,000 U. Proteases activities in the compositions of the present invention can range from about 3,000 to about 500,000 U. In another embodiment, the composition comprises from about 2,000 to about 75,000 USP units of lipase, from about 8,000 to about 250,000 U proteases, and from about 8,000 to about 250,000 U amylases. In yet another embodiment, the composition comprises from about 2,000 to about 40,000 USP units of lipase, from about 8,000 to about 160,000 U proteases, and from about 8,000 to about 160,000 U amylases.

Lipase activities in the compositions can be from about 3000 to about 25,000 IU, from about 4500 to about 25,000 IU, for example from about 4500 to about 5500 IU, from about 9000 to about 11,000 IU, from about 13,500 to about 16,500 IU, and from about 18,000 to about 22,000 IU. Amylase activities in the compositions can be from about 8100 to about 180,000 IU, for example from about 8000 to about 45,000 IU, from about 17,000 to about 90,000 IU, from about 26,000 to about 135,000 IU, from about 35,000 to about 180,000 IU. Protease activities in the compositions can be from about 8000 to about 134,000 IU, for example from about 8000 to about 34,000 IU, from about 17,000 to about 67,000 IU, from about 26,000 to about 100,000 IU, from about 35,000 to about 134,000 IU. In one embodiment, the lipase activity ranges from about 4500 to about 5500 IU, the amylase activity ranges from about 8000 to about 45,000 IU, and the protease activity ranges from about 8000 to about 34,000 IU. In another embodiment, the lipase activity ranges from about 9000 to about 11,000 IU, the amylase activity ranges from about 17,000 to about 90,000 IU, and the protease activity ranges from about 17,000 to about 67,000 IU. In yet another embodiment, the lipase activity ranges from about 13,500 to about 16,500 IU, the amylase activity ranges from about 26,000 to about 135,000 IU, and the protease activity ranges from about 26,000 to about 100,000 IU. In still another embodiment, the lipase activity ranges from about 18,000 to about 22,000 IU, the amylase activity ranges from about 35,000 to about 180,000 IU, and the protease activity ranges from about 35,000 to about 134,000 IU. In still another embodiment, the lipase activity can be about 5,000 or about 30,000 lipase PhEur.

The ratio of amylase/lipase in the compositions can range from about 1.8 to about 8.2, for example from about 1.9 to about 8.2, and about 2.0 to about 8.2. The ratio of protease/lipase in the compositions or oral dosage forms of the present invention can range from about 1.8 to about 6.2, for example about 1.9 to about 6.1, and about 2.0 to about 6.1.

In one embodiment, the ratio of amylase:lipase in the PEP can be in the range of from about 1 to about 10, for example from about 2.38 to about 8.75 (enzymatic assay is performed according to USP). The ratios of protease:lipase in the PEP can be in the range of from about 1.00 to about 8.00, for example from about 1.86 to about 5.13 (enzymatic assay is performed according to USP).

In another embodiment, the activities of lipase, protease, and amylase can be those described in Tables A and B, below:

TABLE A

| | | | | | Ratio | |
|---|---|---|---|---|---|---|
| | | Activity (IU) | | | Amylase/ | Protease/ |
| Formulation | | Lipase | Amylase | Protease | Lipase | Lipase |
| 1 | Min | 4500 | 8100 | 8100 | 1.8 | 1.8 |
| | Max | 5500 | 45000 | 34000 | 8.2 | 6.2 |
| 2 | Min | 9000 | 17100 | 17100 | 1.9 | 1.9 |
| | Max | 11000 | 90000 | 67000 | 8.2 | 6.1 |
| 3 | Min | 13500 | 26100 | 26100 | 1.9 | 1.9 |
| | Max | 16500 | 135000 | 100000 | 8.2 | 6.1 |
| 4 | Min | 18000 | 35100 | 35100 | 2.0 | 2.0 |
| | Max | 22000 | 180000 | 134000 | 8.2 | 6.1 |
| 5 | Min | 3800 | 6800 | 6800 | 1.8 | 1.8 |
| | Max | 4600 | 37700 | 28500 | 8.2 | 6.2 |
| 6 | Min | 9500 | 17100 | 17100 | 1.8 | 1.8 |
| | Max | 11500 | 94300 | 71300 | 8.2 | 6.2 |
| 7 | Min | 15100 | 27200 | 27200 | 1.8 | 1.8 |
| | Max | 18500 | 151700 | 114700 | 8.2 | 6.2 |
| 8 | Min | 18900 | 34000 | 34000 | 1.8 | 1.8 |
| | Max | 23100 | 189400 | 143200 | 8.2 | 6.2 |
| 9 | Min | 5400 | 9700 | 9700 | 1.8 | 1.8 |
| | Max | 6600 | 54100 | 40900 | 8.2 | 6.2 |
| 10 | Min | 10800 | 19400 | 19400 | 1.8 | 1.8 |
| | Max | 13200 | 108200 | 81800 | 8.2 | 6.2 |
| 11 | Min | 21600 | 38900 | 38900 | 1.8 | 1.8 |
| | Max | 26400 | 216500 | 163700 | 8.2 | 6.2 |

TABLE B

| | | | | | Ratio | |
|---|---|---|---|---|---|---|
| | | Activity (PhEur) | | | Amylase/ | Protease/ |
| Formulation | | Lipase | Amylase | Protease | Lipase | Lipase |
| 12 | Min | 9000 | 3900 | 110 | 0.43 | 0.012 |
| | Max | 11000 | 21700 | 2150 | 1.98 | 0.196 |
| 13 | Min | 22500 | 9800 | 280 | 0.43 | 0.012 |
| | Max | 27500 | 54300 | 5400 | 1.98 | 0.196 |
| 14 | Min | 36000 | 15600 | 450 | 0.43 | 0.012 |
| | Max | 44000 | 86900 | 8600 | 1.98 | 0.196 |

The term “U” or “EU” refers to enzymatic units. One USP Unit of amylase activity is contained in the amount of pancrelipase that decomposes starch at an initial rate such that 0.16 μEq of glycosidic linkage is hydrolyzed per minute under the conditions of the Assay for amylase activity from the Official Monograph for Pancrelipase (The 2009 United States Pharmacopeia 32/National Formulary 27) incorporated herein by reference. One USP Unit of lipase activity is contained in the amount of pancrelipase that liberates 1.0 μEq of acid per minute at pH 9.0 and 37° C. under the conditions of the Assay for lipase activity from the Official Monograph for Pancrelipase (The 2009 United States Pharmacopeia 32/National Formulary 27) incorporated herein by reference. One USP Unit of protease activity is contained in the amount of pancrelipase that under the conditions of the Assay for protease activity from the Official Monograph for Pancrelipase (The 2009 United States Pharmacopeia 32/National Formulary 27) incorporated herein by reference, hydrolyzes casein at an initial rate such that there is liberated per minute an amount of peptides not precipitated by trichloroacetic acid that gives the same absorbance at 280 nm as 15 nmol of tyrosine.

Below is a table for converting units of amylase, lipase, and protease.

Conversion values for units of enzyme activity

| | | | | |
|---|---|---|---|---|
| Amylase | 1 PhEur unit | equals 1 FIP unit | equals 1 BP unit | equals 4.15 USP units |
| Lipase | 1 PhEur unit | equals 1 FIP unit | equals 1 BP unit | equals 1 USP unit |
| Protease | 1 PhEur unit | equals 1 FIP unit | equals 1 BP unit* | equals 62.5 USP units |

*Only free protease for pancreatin; total protease for pancreatic extract.
BP—British Pharmacopoeia;
FIP—Federation Internationale Phannaceutique;
PhEur—European Phamacopoeia

The total amount of digestive enzymes (by weight) in the compositions or oral dosage forms of the present invention can be from about 65 to about 100%, from about 80 to about 100%, from about 90 to about 100%, from about 95 to about 100 or about 85%, about 90%, about 95%, or about 100%, inclusive of all ranges and subranges therebetween. In one embodiment, the total amount of digestive enzymes is from about 80 to about 100%. In another embodiment, the total amount of digestive enzymes (e.g., pancrelipase) ranges from about 90 to about 99% (e.g., about 98%).

In one embodiment the dosage forms of the present invention comprise at least one digestive enzyme, have a moisture content of about 10% or less, about 5% or less, about 3% or less, about 2.5% or less, about 1.5% or less, or about 1% or less, inclusive of all ranges and subranges therebetween (e.g., any of about 2.5% to about 3%, about 2% to about 3%, about 1.5% to about 3%, about 1% to about 3%, about 2% to about 2.5%, about 1.5% to about 2.5%, about 1% to about 2.5%, about 1.5% to about 2%, about 1% to about 2%, and about 1% to about 1.5%). Compositions maintained at low moisture content have been found to be substantially more stable compared to conventional compositions maintained at higher moisture contents, e.g. above about 3% or higher. Moisture content can be measured by loss on drying (LoD) USP method.

In yet another embodiment, the compositions exhibit a loss of enzyme activity measured in the inner core of the composition of no more than about 25%, no more than about 20%, no more than about 15%, no more than about 12%, no more than about 10%, no more than about 8%, or no more than about 5%, after being submerged in simulated acidic solution for 4 hour at room temperature.

The term “simulated gastric fluid” (or SGF) refers to a gastric fluid solution prepared as follows: Dissolve 2.0 g of sodium chloride in 7.0 mL of hydrochloric acid and sufficient water to make 1000 mL. This test solution has a pH of about 1.2. See U.S. Pharmacopeia 29^{th} Ed., Test Solutions, Simulated Gastric Fluid.

The term “simulated intestinal fluid” (or SIF) refers to an intestinal fluid solution prepared as follows: Dissolve 6.8 g of monobasic potassium phosphate in 250 mL in water, mix, and add 77 mL of 0.2 N sodium hydroxide and 500 mL of water. Adjust the resulting solution with either 0.2 N sodium hydroxide or 0.2 N hydrochloric acid to a pH of 6.8±0.1. Dilute with water to 1000 mL. See US. Pharmacopeia 29^{th} Ed., Test Solutions, Simulated Intestinal Fluid.

The compositions of the present invention can be prepared into or incorporated into any suitable oral dosage form. Non-limiting examples of suitable dosage forms include tablets or multiparticulates (such as mini-tablets, micro-tablets, and prills), for which one or multiple units can be eventually incorporated into, for example, a capsule. In a preferred embodiment, the pharmaceutical composition is in the form of tablets. In a more preferred embodiment, the tablet is free of or substantially free of excipients and is not enterically coated.

The composition (e.g., a mini-tablet or tablet) can have a diameter ranging from about 0.5 to about 15 mm, from about 2 to about 10 mm, or from about 4 to about 10 mm. For example, the diameter can be about 2, about 4, about 6, about 8, about 9.7, or about 10 mm. The tablet diameter can be measured, for example, with a caliper.

The term “excipient” refers to any inert substance added to a pharmaceutical composition. Non-limiting examples of excipients include those excipients described in the Handbook of Pharmaceutical Excipients. American Pharmaceutical Association, 6^{th} Ed. (2009). Excipients can include, for example, fillers such as saccharides, for example, lactose or sucrose, mannitol or sorbitol, cellulose preparations and/or calcium phosphates, for example, tricalcium phosphate or calcium hydrogen phosphate, binders, such as, starch, using, for example, maize starch, wheat starch, rice starch, potato starch, gelatin, methyl cellulose, hydroxy-propylmethylcellulose, sodium carboxymethylcellulose, and/or polyvinyl pyrrolidone, and/or polyethylene glycol, auxiliaries such as flow-regulating agents, and lubricants, for example, silica, talc, and/or stearic acid or salts thereof, such as magnesium stearate or calcium stearate.

The term “coating”, as used herein, refers to a material used to coat a formed composition (e.g., tablet), typically for the purpose of protecting the active ingredient or drug substance present in the composition against degradation, to provide a desired release pattern for the drug substance after administration, to mask the taste or odor of the drug substance, or for aesthetic purposes. The coating may consist of for example, sugar coating, film coating, or enteric coating. Sugar coating is water-based and results in a thickened covering around a formed tablet. A film coat is a thin cover around a formed tablet or bead. Unless it is an enteric coat, the film coat will dissolve in the stomach. An enteric-coated tablet or bead will pass through the stomach and break up in the intestines. Water-insoluble coatings comprising, for example, ethylcellulose, may be used to coat tablets and beads to slow the release of drug as the tablet passes through the gastrointestinal tract. Hydroxyethyl cellulose, hydroxypropyl cellulose, hydroxypropyl methylcellulose, methylcellulose and ethylcellulose are examples of film coatings. Enteric coatings may comprise, for example, cellulose acetate phthalate, shellac, methacrylate polymers, and alginate.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms. The term “mammal” includes human subjects.

The following examples are given as specific illustrations of the invention. It should be understood, however, that the invention is not limited to the specific details set forth in the examples. All parts and percentages in the examples, as well as in the remainder of the specification are by weight unless otherwise specified.

Further, any range of numbers recited in the specification or paragraphs hereinafter describing or claiming various aspects of the invention, such as that representing a particular set of properties, units of measure, conditions, physical states, or percentages, is intended to literally incorporate expressly herein by reference or otherwise, any number falling within such range, including any subset of numbers or ranges subsumed within any range so recited.

### EXAMPLES

### Example 1

### Excipient-Free Digestive Enzyme Tablet

TABLE I
Excipient-Free Tablet (500 mg tablet)
(Obtained by Direct Compaction at 2.5T)

| Tablet Components | Amount |
|---|---|
| Lipase | 25,000 USP Units |
| Amylase | 94,000 USP Units |
| Protease | 94,000 USP Units |

Excipient-free tablets were prepared by direct compression of 500 mg of active substance (having the enzymatic activity for lipase, proteases and amylase as mentioned in Table 1) in a die with a diameter of 9.7 mm.

Smaller tablets as indicated below were also prepared. Each size of smaller tablets were prepared in sufficient number such that their total had an overall mass close to 500 mg. (equivalent to one 9.7 mm tablet).
Tablet 2.0 mm (34 mini-tablets)
Tablet 4.0 mm (8 tablets)
Tablet 6.0 mm (4 tablets)
Tablet 9.7 mm (1 tablet)

### Example 2

### Evaluation of Excipient-Free Pancreatic Enzyme Tablet and Reference Tablet Containing 40% w/w Excipients in Simulated Gastric Fluid and Simulated Intestinal Fluid

The enzyme activity of the excipient-free tablets of Example 1 and reference uncoated tablets containing excipients was evaluated in Simulated Gastric Fluid (SGF) and Simulated Intestinal Fluid (SIF) as described below. The reference tablets contained 8,000 USP units of lipase, 30,000 USP units of amylase, and 30,000 USP units of proteases and approximately 40% w/w of pharmaceutical excipients. The reference tablets were prepared by direct compression. The results are shown in Tables II-V.

### Methods

Tablets were maintained in a solution of SGF (50 mL) at pH 1.2 or SIF (50 mL) at pH 6.8 at room temperature with constant rotatory stirring (50 rpm). Lipase, amylase, and proteases activities of each sample were measured over time using the inner part of the tablets (i.e., a part of the tablet that was still dry and not hydrated by the dissolution media). Evaluation was done using the pancrelipase USP monographed methods for all three enzymes.

### Results

The excipient-free tablets maintained significant lipase, amylase and protease activity following exposure to the simulated gastric and intestinal fluids. Specifically, 92.5% of lipase activity and 41.83% amylase activity was maintained in excipient-free tablets exposed to SGF for 2 hours. 79.16% protease activity was observed in the excipient-free tablets immersed in SGF for 1 hour followed by 0.5 hour in SIF.

Low levels of enzyme activity were retained in the reference tablets where the active principle was mixed with pharmaceutical acceptable excipients. In the presence of intestinal fluids, the reference tablets exhibited an activity loss exceeding 75% activity for each of lipase, proteases and amylase.

TABLE II
COMPARATIVE EVALUATION OF LIPASE ACTIVITY OF
EXCIPIENT-FREE TABLET AND REFERENCE TABLET

| | Percent Activity | |
|---|---|---|
| | (relative to initial activity) | |
| Dissolution conditions | Example 1 | Reference tablet |
| Initial Activity | 100 | 100 |
| Activity After Exposure To SGF, for | 87.1 | 0 |
| 1 Hr | | |
| Activity After Exposure To SGF for | 92.5 | 0 |
| 2 Hrs | | |
| Activity After Exposure To SGF, for | 94.4 | 0 |
| 1 hrs and SIF, for 0.5 Hr | | |

TABLE III
COMPARATIVE EVALUATION OF PROTEASES ACTIVITY OF
EXCIPIENT-FREE TABLET AND REFERENCE TABLET

| | Percent Activity | |
|---|---|---|
| | (relative to initial activity) | |
| Dissolution conditions | Example 1 | Reference tablet |
| Initial Activity | 100 | 100 |
| Activity After Exposure To SGF, | 75.17 | 7.05 |
| for 1 Hr | | |
| Activity After Exposure To SGF for | 84.1 | 15.03 |
| 0.5 Hrs | | |
| Activity After Exposure To SGF, for 1 hr | 79.16 | 7.68 |
| and SIF, for 0.5 Hr | | |

TABLE IV
COMPARATIVE EVALUATION OF AMYLASE ACTIVITY OF
EXCIPIENT-FREE TABLET AND REFERENCETABLET

| | Percent Activity | |
|---|---|---|
| | (relative to initial activity) | |
| Dissolution conditions | Example 1 | Reference tablet |
| Initial Activity | 100 | 100 |
| Activity After Exposure To SGF, for | 70.79 | 11.42 |
| 1 Hr | | |
| Activity After Exposure To SGF for | 41.83 | 0 |
| 2 Hrs | | |
| Activity After Exposure To SGF, for | 80.28 | 13.83 |
| 1 hr and SIF, for 0.5 Hr | | |
| Activity After Exposure To SIF, for | 86.25 | 34.20 |
| 0.5 Hr | | |

### Example 3

### Evaluation of Lipase Activity Determined on the Entire Tablet after Exposure to SGF at Various Time Intervals

Tablets prepared in Example 1 and Reference Tablets as described in Example 2 were exposed to SGF for 30, 60, and 120 minutes, and the lipase activity of the entire resulting tablets were evaluated. The results are shown in Table V below.

TABLE V
EXCIPIENT-FREE TABLET CONTAINING 500 mg TABLET
OBTAINED BY DIRECT COMPACTION AT 2.5T

| | Remaining lipase activity after exposure to SGF | | |
|---|---|---|---|
| | (% reported to the initial value) | | |
| Dosage form | 30 min in SGF | 60 min SGF | 120 min SGF |
| Excipient-free tablet | 64.35 ± 4.17 | 44.24 ± 2.7 | 22.90 ± 3.9 |
| (example 1) | | | |
| Reference tablet | 0.0 | 0.0 | 0.0 |
| (containing | | | |
| excipients) | | | |

### Example 4

### Evaluation of Friability and Hardness of Excipient-Free Tablet Digestive Enzyme Compositions

Excipient-free tablets were prepared as described in Example 1 and were compacted using compression forces of 1.0-3.0 T. The friability and hardness of each tablet were measured. The results are provided in Table VI.

### Methods

All tablets were prepared 24 hours before testing hardness and friability.

Tablet hardness (kp was measured using an automatic tablet hardness tester (model TBH 30, Erweka). The results reported represent an average of 5 measurements with 10 tablets each.

Tablet friability was determined using standard methods with an automatic friabilator. Percent friability of each tablet was calculated from the amount of tablet weight loss due to instrument rotation cycles as indicated in USP method no. 1216. The reported results represent an average of 5 measurements.

### Results

The friability of excipient-free tablets was shown to decrease with increasing compression force used to prepare the tablets. The hardness for the tablets increased with increasing tablet compression force. Suitable friability and hardness are met in tablets prepared using a compression force of 1.0-3.0 T.

TABLE VI
EVALUATION OF FRIABILITY AND HARDNESS OF
EXCIPIENT-FREE TABLET

| FORMULATION/ | | |
|---|---|---|
| COMPRESSION | FRIABILITY | HARDNESS |
| FORCE (T) | (%) | (kp) |
| Example 1 (1.0 T) | 8.33 | Mean 5.3 |
| Example 1 (1.5 T) | 0.35 | Mean 7.0 |
| Example 1 (2.0 T) | 0.30 | Mean 8.4 |
| Example 1 (2.5 T) | 0.20 | Mean 8.3 |
| Example 1 (3.0T) | 0.20 | Mean 10.1 |
| Reference tablets | 0.11 | Mean 11.7 |

### Example 5

### Mechanical Behavior of Excipient-Free Tablets Exposed to SGF

The mechanical behaviour of the excipient-free tablets submerged in SGF is shown in Table VII.

### Methods

The excipient-free tablets were prepared as described in Example 1 and were compressed using two compression force ranges (A: 1.0-2.5 T and B 2.5-5.0 T).

Tablets were suspended in a solution of SOF (50 mL) at pH 1.2 for 30, 60, and 120 minutes followed by exposure for 0, 30, 60, and 120 minutes in SIF (50 mL) at pH 6.8 at room temperature with constant stirring (50 rpm). Table VII shows treatment with SGF at different times followed by SIF.

### Results

Complete disintegration of the excipient-free tablets occurred after subjecting tablets to SGF with subsequent exposure with SIF for 120 minutes. During dissolution, tablet swelling and erosion of the external layer was observed. SIF clearly accelerated the erosion/dissolution which is useful for intestinal delivery.

TABLE VII
BEHAVIOUR OF EXCIPIENT-FREE TABLET COMPRESSED AT A: 1.0-2.5 T OR
B: 2.5-5.0 T AND EXPOSED TO SIMULATED GASTRIC AND SIMULATED
INTESTINAL FLUIDS FOR VARIOUS INTERVALS OF TIME

| Time tablets | Time tablets | Residue of tablets | Residue of tablets |
|---|---|---|---|
| were exposed to | were exposed to | compacted at force range A | compacted at force range B |
| SGF | SIF | (%) | (%) |
| 30 min | 0 min | 90% of initial tablet | 90% of initial tablet |
| | 30 min | 80% of initial tablet | 80% of initial tablet |
| | 60 min | 20% of initial tablet | 50% of initial tablet |
| | | (tablet deformation and | |
| | | fragility observed) | |
| | 120 min | tablet is completely | tablet is completely |
| | | disintegrated | disintegrated |
| 60 min | 0 min | 80% of initial tablet | 80% of initial tablet |
| | 30 min | 40% of initial tablet | 50% of initial tablet |
| | 60 min | 20% of initial tablet | 30% of initial tablet |
| | | (tablet deformation and | (tablet deformation and |
| | | fragility observed) | fragility observed) |
| | 120 min | tablet is completely | tablet is completely |
| | | disintegrated | disintegrated |
| 120 min | 0 min | 60% of initial tablet | 70% of initial tablet |
| | 30 min | 20% of initial tablet | 25% of initial tablet |
| | 60 min | 10% of initial tablet | 10% of initial tablet |
| | | (tablet deformation and | (tablet deformation and |
| | | fragility observed) | fragility observed) |
| | 120 min | tablet is completely | tablet is completely |
| | | disintegrated | disintegrated |

### Example 6

### Evaluation of Gastric Stability

Excipient-free tablets were prepared as described in Example 1 and were compressed using two compression force ranges (A: 1.0-2.5 T and B 2.5-5.0 T).

### Methods

Tablets were submerged in SOF (800 mL) at pH 1.2 at 37° C. with constant stirring (100 rpm) using an USP apparatus 2. Lipase activity of the entire tablet was monitored over a 120 minute time interval. Reference tablets as described in Example 2 were also evaluated.

### Results

Significant lipase activity was maintained in excipient-free tablets exposed to SGF at 60 minute and 120 minute time intervals, as shown in Table VIII.

TABLE VIII
COMPARATIVE EVALUATION OF LIPASE ACTIVITY FROM
EXCIPIENT-FREE TABLET AND REFERENCE TABLET

| | Activity reported to | Activity |
|---|---|---|
| | initial lipase activity | reported to initial lipase |
| Formulation/ | after 60 minutes of | activity after120 minutes |
| Compression Force | exposure to SGF (%) | of exposure to SGF (%) |
| Example 1 | 50.88 | 29.37 |
| Compression | | |
| range A | | |
| Example 1 | 55.50 | 38.94 |
| Compression | | |
| range B | | |
| Reference tablet | 0.0 | 0.0 |

### Example 7

### Evaluation of Flow Properties of Powders

Excipient-free tablets were prepared as described in Example 1. The flowability scale, including the compressibility index, flow character, and Hausner ratio, of the tablets was determined according to the procedures outlined in the U.S. Pharmacopeia (USP29<1174>) (www.pharmacopeia.cn/v29240/-usp29nf24s0_c1174.html). The results are shown in Table IX.

TABLE IX
SCALE OF FLOWABILITY (theoretical values as per USP 29)

| COMPRESSIBILITY | | |
|---|---|---|
| INDEX (%) | FLOW CHARACTER | HAUSNER RATIO |
| ≦10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very poor | 1.46-1.59 |
| ≧38 | Very, very poor | ≧1.60 |

### Results

When compared with theoretical values found in the flowability scale (Table IX), pancreatic enzyme concentrate (PEC) powders exhibited suitable flowability as indicated by their compressibility index and Hausner ratio data. In an additional experiment, a pharmaceutical excipient (i.e. stearic acid) was incorporated into the enzyme powder used in Example 1, and the compressibility index, Hausner ratio, and flow character were evaluated. It can be concluded that at a 2% level the lubricant did not significantly change the flowability and compressibility characteristics of proposed powders.

TABLE X
FLOWABILITY OF PEC POWDER WITH AND WITHOUT
STERIC ACID

| | COMPRESSIBILITY | HAUSNER | FLOW |
|---|---|---|---|
| FORMULATION | INDEX (%) | RATIO | CHARACTER |
| PEC powder | 25 | 1.33 | Passable |
| PEC prepared | 24 | 1.31 | Passable |
| with 2% steric | | | |
| acid | | | |

### Example 8

Tablets were prepared by the procedure described in Example 1 having the weights indicated in Table XI. The hardness of the tablets prior SGF exposure and the lipase activity of the tablets after exposure SOF were measured. The results are shown in Table XI.

TABLE XI
CHARATERISTICS OF EXCIPIENT-FREE TABLETS OF VARIOUS
SIZE AND THEIR GASTRORESISTANCE AFFORDED WHEN
EXPOSED FOR DIFFERENT INTERVALS IN SGF

| | | Lipase activity | | |
|---|---|---|---|---|
| | | (%)* in the | | |
| | Tablet | residual tablet | | |
| Weight/unit | Hardness | after various | | |
| (average) | (kp) | exposure times | | Observations |
| 15 mg | Mean 2.3 | 0.5 h | Not | External thin layer is |
| | | 1.0 h | detectable | formed the internal part of |
| | | 2.0 h | | the tablet (core) becomes |
| | | | | wet |
| 64 mg | Mean 1.0 | 0.5 h | 37.1% | External thin layer is |
| | | 1.0 h | 16.6% | formed and the internal part |
| | | 2.0 h | 0% | of the tablet (core) remains |
| | | | | dry |
| 142 mg | Mean 1.0 | 0.5 h | 46.6% | External thin layer is |
| | | 1.0 h | 31.4% | formed and the internal part |
| | | 2.0 h | 13.0% | of the tablet (core) remains |
| | | | | dry |
| 500 mg | Mean 9.3 | 0.5 h | 67.8% | External thin layer is |
| | | 1.0 h | 52.4% | formed and the internal part |
| | | 2.0 h | 36.8% | of the tablet (core) remains |
| | | | | dry |

*Percentage in comparison with the initial value of lipase activity in PEC used for tablets.
The USP apparatus 1 was used for dissolution.
All tablets were compressed at 2T

### Example 9

### Hydration Kinetics of Excipient-Free Tablet in SGF

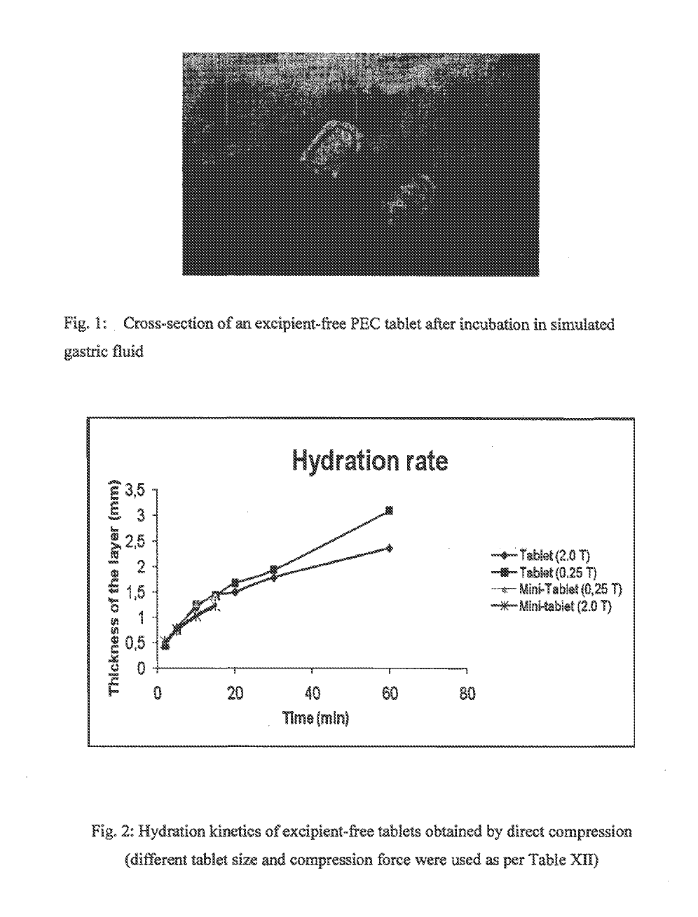

Tablets were prepared by the procedure described in Example 1 having the sizes indicated in Table XII. The thickness of the hydrated layer after exposure to SGF was measured. The results are shown in Table XII and FIG. 2. An image of the hydrated layer formed in one tablet is shown in FIG. 1.

TABLE XII

| | Hydrated layer thickness (mm) | | | |
|---|---|---|---|---|
| | | Excipient-free | Excipient-free | |
| | Excipient-free | tablet | tablet | Excipient-free |
| Time | tablet | Diameter | Diameter | tablet |
| In | Diameter | 9.7 mm | 2.0 mm | Diameter |
| SGF | 9.7 mm | Compaction | Compaction | 2.0 mm |
| (min) | Compaction 2T | 0.25T | 0.25T | Compaction 2T |
| 2 | 0.45 | 0.45 | 0.55 | 0.50 |
| 5 | 0.79 | 0.75 | 0.75 | 0.75 |
| 10 | 1.20 | 1.25 | 1.27 | 1.03 |
| 15 | 1.45 | 1.44 | 1.42 | 1.24 |
| 20 | 1.50 | 1.68 | — | — |
| 30 | 1.79 | 1.94 | — | — |
| 60 | 2.37 | 3.10 | — | — |

### Example 10

### Lipase Recovery in SIF from Excipient-Free Tablets after Exposure for 1 H in SGF

Tablets were prepared by the procedure described in Example 1 having the weights indicated in Table XIII. All tablets were compressed at a compression force range A. The lipase activity in the tablet after exposure to SGF and subsequent exposure to SIF was evaluated in dissolution medium. The results are shown in Table XIII below.

TABLE XIII

| | Lipase activity | Lipase activity in pH 6 after 1 hour in SGF |
|---|---|---|
| Tablet | after 1 h in SGF | followed by the indicated minutes in SIF |
| mass | (App. I) | (App. II) |
| 15 mg | Not detectable | NA |
| 64 mg | 16.6%* | after 10 min 330 IU (6%)* |
| | | after 20 min 340 IU |
| | | after 30 min 340 IU |
| 142 mg | 31.4%* | after 10 min 1024IU (8%)* |
| | | after 20 min 1040 IU |
| | | after 30 min 1057 IU |
| 500 mg | 52.4%* | after 15 min 5 625 IU (12.5%)* |
| | | after 30 min 7 155 IU (15.9%)* |
| | | after 60 min 9 405 IU (20.9%)* |

*Percentage from the initial lipase activity in the PEC used for tablet preparation

### Example 11

### Lipase Activity of Excipient-Free Tablets after Exposure Mimicking the In Vivo Conditions

Tablets were prepared by the procedure described in Example 1 having the weights indicated in Table XIV. All tablets were obtained by direct compression at a compression force ranging between 1-2.5 T (range A). The lipase activity in the tablet after exposure to SGF (pH=1.2) for 1 hour, exposure to fluid at a pH of 4.5 for 1 hour, and subsequent exposure to SIF for 15 minutes was evaluated. The results are shown in Table XIV below.

TABLE XIV

| | | Lipase activity after 1 |
|---|---|---|
| | Lipase activity | hour in SGF, 1 h at pH |
| | (dosage in the residual | 4.5 (App. I) and 15 min |
| | tablet) after 1 h in | in SIF |
| Mass/dosage unit | SGF | (disintegration) |
| 15 mg | Not detectable | NA |
| 64 mg | 16.60% | 13.27%* |
| 142 mg | 31.40% | 21.20%* |
| 500 mg | 53.50% | 42.39%* |

*Percentage from the initial lipase activity in the PEC used for tablet preparation

All patents and patent applications cited in this specification are incorporated herein by reference in their entirety and to the same extent as if each reference was individually incorporated by reference.

1. A gastroresistant compacted pharmaceutical composition comprising one or more enzymes self-assembled such that the enzymes have greater cohesive strength after compaction than prior to compaction, wherein the enzymes in the pharmaceutical composition retain at least 30% of their activity after exposure of the pharmaceutical composition to simulated gastric fluid for 1 hour at 37° C.

2. The composition of claim 1, wherein the composition becomes self-coated in situ upon contact with gastric fluids limiting further penetration of the fluid.

3. The composition of claim 1, wherein the composition is a tablet.

4. (canceled)

5. (canceled)

6. The composition of claim 1, wherein the composition comprises one or more enzymes selected from amylases, lipases, and proteases.

7. The composition of claim 1, wherein the composition comprises pancrelipase.

8. The composition of claim 1, wherein the composition has a drug content of at least 65% by weight.

9. (canceled)

10. (canceled)

11. The composition of claim 1, wherein the composition has a drug content of at least 95% by weight.

12. (canceled)

13. The composition of claim 1, wherein the composition is not enterically coated.

14. The composition of claim 1, wherein the composition is monolithic.

15. The composition of claim 1, wherein the composition is blended together with enterically coated pancrelipase compositions.

16. (canceled)

17. A monolithic, compacted, gastro-resistant pharmaceutical composition comprising pancrelipase, the pancrelipase comprising a mixture of lipase, amylase, and protease, wherein the lipase and amylase in the tablet retain at least 80% and 30% of their activity, respectively, after exposure to simulated gastric fluid for 2 hours, and the protease in the tablet retains at least 70% of its activity after exposure to simulated gastric fluid for 0.5 hours.

18. (canceled)

19. (canceled)

20. (canceled)

21. (canceled)

22. (canceled)

23. (canceled)

24. (canceled)

25. (canceled)

26. The monolithic, compacted, gastro-resistant pharmaceutical composition of claim 17 wherein the pancrelipase self-assembles so as to enhance cohesion within the composition.

27. The monolithic, compacted, gastro-resistant pharmaceutical composition of claim 17, wherein the composition becomes coated in situ upon contact with gastric fluid.

28. (canceled)

29. The composition of claim 1, wherein the composition is substantially free of excipients and is not enterically coated.

30. (canceled)

31. (canceled)

32. (canceled)

33. (canceled)

34. The composition of claim 29, wherein the dosage form comprises pancrelipase.

35. (canceled)

36. (canceled)

37. The composition of claim 29, wherein the composition is free of excipients.

38. The composition of claim 1, wherein the composition is multi-layered, and one or more enzymes are in an outermost layer of the composition.

39. (canceled)

40. (canceled)

41. The composition of claim 38, wherein the composition is blended together with enterically coated pancrelipase dosage forms.

42. (canceled)

43. (canceled)

44. A process for preparing a pharmaceutical composition comprising one or more enzymes, the method comprising compacting an enzyme preparation free or substantially free of excipients.

45. The process of claim 44, wherein the compaction is performed at a compression force of from about 0.25 T to about 3.0 T.

46. A method for treating a digestive disorder comprising administering to a patient in need thereof a composition of claim 1.

47. The method of claim 46, wherein the patient suffers from partial or complete exocrine pancreas insufficiency, and the composition comprises pancrelipase.

48. The method of claim 47, wherein the exocrine pancreas insufficiency is concomitant with cystic fibrosis, chronic pancreatitis, post-pancreatectomy, post-gastrointestinal bypass surgery, ductal obstruction from neoplasm, alcoholism, or pancreatic carcinomas.

49. A method of controlling steatorrhea comprising administering to a patient in need thereof a composition of claim 1, wherein composition comprises pancrelipase.
